(19) United States
(12) Patent Application Publication
(10) Pub. No.: US 2025/0257032 A1
(43) Pub. Date: Aug. 14, 2025

(54) METHOD OF PREPARING HIGH PURITY LUTEIN ESTER CRYSTALS, AND APPLICATIONS THEREOF

(71) Applicant: AVT NATURAL PRODUCTS LTD., Chennai (IN)

(72) Inventors: Rahul THOMAS, Chennai (IN); Krishna Kumar BALASUNDARAM, Chennai (IN); Pushpakumari Kaliyarmattom NARAYANAN NAIR, Aluva (IN); Naijo VARGHESE, Aluva (IN); Pramod SIVADASAN, Aluva (IN); Deepak Padinjaroot BALAKRISHNAN, Aluva (IN)

(21) Appl. No.: 19/049,350

(22) Filed: Feb. 10, 2025

(30) Foreign Application Priority Data
Feb. 9, 2024 (IN) 202441008970

(51) Int. Cl. C07C 403/24; C07D 311/22

(52) U.S. Cl. CPC C07C 403/24; C07D 311/22; C07B 2200/13

(57) ABSTRACT

The present disclosure provides a method for preparing lutein ester, by providing a raw material selected from marigold flowers or marigold meal; subjecting the raw material to extraction using ethanol followed by acetone; and concentrating to obtain an extract which may then be crystallized to obtain lutein ester crystal of high purity. Further, the present disclosure provides a method for preparing lutein ester, by providing a raw material selected from marigold flowers or marigold meal; subjecting the raw material to extraction using isopropanol; and concentrating to obtain an extract which may then be crystallized to obtain lutein ester crystal of high purity.

### FIELD OF INVENTION

The present disclosure broadly relates to the field of carotenoids, particularly lutein esters and lutein, and their preparation methods. The present disclosure particularly relates to a method for preparing lutein ester crystals.

### BACKGROUND OF INVENTION

The major carotenoids in marigold flower petals are xanthophylls, along with minor quantities of carotenes such as beta carotene and beta cryptoxanthin, and plant waxes. Marigold flower petals are a rich source of xanthophylls which majorly constitute lutein esters, and smaller quantities of zeaxanthin esters. Both, lutein and zeaxanthin are present as mono and diesters of lauric, myristic, palmitic and stearic acids. Lutein ester obtained from such sources may be subjected to enzymatic hydrolysis or alkali hydrolysis, for producing free lutein. Lutein is used in feed compositions, as a colouring agent, and as a nutraceutical supplement in foods.

Recent studies have shown that lutein esters have high or higher bioavailability as compared to free lutein (for e.g. refer WO1998045241A2). Hence, there exists a need for producing lutein esters, which are a more natural form of lutein. Current processes to extract lutein esters include multiple steps with lower xanthophyll recoveries, lower product yield and are expensive, involving extraction using solvents such as hexane, tetrahydrofuran, sub or supercritical fluids, etc. Such processes include the use of marigold oleoresin obtained from marigold flowers, which is further processed to obtain lutein esters, making it an energy intensive and cumbersome process.

For example, U.S. Pat. No. 4,048,203A discloses a method involving the use of marigold oleoresin which is processed using a hot alkyl-alcohol, to obtain about 51% lutein fatty acid ester.

CN1432567A discloses a method of producing lutein ester from marigold oleoresin by extraction with acetone, wherein the acetone extract obtained is concentrated and crystallized using butanol or isopropanol, however the yield of the obtained product is low.

EP1857441B1 discloses a method of separating and purifying xanthophyll fatty acid ester from marigold oleoresin involving multiple extraction steps using extractants comprising n-hexane. Hence, there exists a need for developing processes that are simple, cost effective, and efficient, for producing lutein esters of high purity.

### SUMMARY OF THE INVENTION

In an aspect of the present disclosure, there is provided a method for preparing lutein ester, comprising: (a) providing a raw material selected from shredded marigold flowers, marigold meal, or combination thereof, (b) subjecting the raw material to extraction with at least one polar solvent selected from ethanol, methanol, or combination thereof, to obtain a polar solvent fraction and residual biomass; (c) subjecting the residual biomass to extraction with an extractant comprising acetone, to obtain an acetone fraction; and (d) concentrating the acetone fraction.

In an aspect of the present disclosure, there is provided a method for preparing lutein ester, said method comprising: (a) providing a raw material selected from shredded marigold flowers, marigold meal, or combination thereof, (b) subjecting the raw material to extraction with an extractant comprising isopropanol, to obtain an isopropanol fraction; and (d) concentrating the isopropanol fraction, and optionally followed by crystallization to obtain lutein ester crystals.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification and examples are delineated here. These definitions should be read in the light of the remainder of the disclosure and understood by a person of skill in the art. The terms used herein have the meanings recognized and known to those of skill in the art. However, for convenience and completeness, particular terms and their meanings are set forth below.

The articles “a”, “an” and “the” are used to refer to one or to more than one (i.e., to at least one) of the grammatical objects of the article.

Throughout this specification, unless the context requires otherwise, the word “comprise” and variations such as “comprises” and “comprising” are used in the inclusive, open sense and will be understood to imply the inclusion of a stated element or step or group of elements or steps but not the exclusion of any other element or step or group of elements or steps. It is not intended to be construed as “consists of only”.

The term “including”, as used herein, means “including but not limited to”. “Including” and “including but not limited to” are used interchangeably.

The term “xanthophyll”, as used herein, broadly refers to carotenoids, and/or ester thereof. Examples include lutein, zeaxanthin, lutein esters, zeaxanthin esters, beta carotene, cryptoxanthin, etc.

The term “lutein”, as used herein, refers to a carotenoid that forms a major constituent of xanthophylls present in marigold flowers, particularly petals. The term “lutein ester”, as used herein, refers to lutein mono and/or diesters, for eg: mono and/or diesters of lauric, myristic, palmitic and stearic acids.

The term “zeaxanthin”, as used herein, refers to a carotenoid that forms a minor constituent of xanthophylls present in marigold flowers, particularly petals. The term “zeaxanthin ester”, as used herein, refers to zeaxanthin mono and/or diesters, for eg: zeaxanthin mono and/or diesters of lauric, myristic, palmitic and stearic acids.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods and materials are now described.

The present disclosure is not to be limited in scope by the specific embodiments described herein, which are intended for the purposes of exemplification only. Functionally equivalent products, compositions, and methods are clearly within the scope of the disclosure, as described herein.

As discussed in the background, currently known methods involve the use of marigold oleoresin for preparation of lutein esters. Such methods involve the use of extractants such as n-hexane, tetrahydrofuran, and sub or supercritical fluids. For pharmaceutical and nutraceutical applications of lutein esters, the use of such solvents may not be preferred. Further, n-hexane is a non-selective solvent, which can also extract undesired lipophilic compounds, such as fatty acids, and other carotenoids, potentially compromising the purity of lutein ester extracts from marigold flowers. To achieve higher purity, further purification techniques, such as enrichment, chromatography, etc. are typically required to separate lutein esters from these co-extracted substances. Hence, isolation of lutein ester with high purity and good recovery has always been challenging. Additionally, alcohol-based solvents, particularly ethanol, align with green chemistry principles, is more sustainable and environmentally friendly compared to n-hexane. Accordingly, the inventors of the present invention have provided a simple, cost-effective, and efficient process involving the use of marigold flowers or marigold meal, directly, as raw material. According to the method as disclosed herein. Thus, minimizing the number of processing steps while achieving high purity lutein esters crystals with good yield, and higher recovery percentage. The process comprises lesser number of unit operations and with increased product recoveries.

Embodiments herein provide a method for preparing lutein ester. The method, according to embodiments herein, is capable of yielding lutein ester in high purity. In an embodiment, the method comprises: (a) providing a raw material selected from shredded marigold flowers, marigold meal, or combination thereof; (b) subjecting the raw material to extraction with at least one polar solvent selected from ethanol, methanol, or combination thereof, to obtain a polar solvent fraction and residual biomass; (c) subjecting the residual biomass to extraction with an extractant comprising acetone, to obtain an acetone fraction; and (d) concentrating the acetone fraction, or concentrating the acetone fraction to obtain an extract. In an embodiment, the method comprises: (a) providing a raw material selected from shredded marigold flowers, marigold meal, or combination thereof, (b) subjecting the raw material to extraction with at least one polar solvent selected from ethanol, methanol, or combination thereof, to obtain a polar solvent fraction and residual biomass; (c) subjecting the residual biomass to extraction with an extractant comprising acetone, to obtain an acetone fraction; and (d) concentrating the acetone fraction to obtain an extract comprising lutein ester, followed by crystallization.

The method for preparing lutein ester, according to embodiments herein, includes the use of raw material selected from shredded marigold flowers, marigold meal, or combination thereof. The raw material, according to embodiments herein, may be in the form of shredded marigold flowers, marigold meal, or combination thereof. Accordingly, in an embodiment, the raw material is selected from shredded marigold flowers, marigold meal, or combination thereof. Marigold, according to the present disclosure, refers to a plant of the genus Tagetes, particularly Tagetes erecta. In an embodiment, marigold refers to the flower of the plant of the genus Tagetes. Marigold flowers comprise of various natural compounds, such as terpenes, xanthophylls, and flavonoids.

In a preferred embodiment, the marigold seeds (seeds sourced from Thailand) are grown in suitable conditions for flowering, and then the flowers are collected for lutein ester extraction and preparation according to embodiments herein.

The term “shredded marigold flowers”, as used herein, refers to fresh flowers, that are preferably silaged, de-juiced, and shredded. De-juicing is performed to remove the water content from the silaged flowers, preferably by mechanical pressing. The flowers are silaged by subjecting the flowers to silaging process. The term “silaging”, as used herein, refers to a process of subjecting a plant material to anaerobic bacterial fermentation. Silaged flowers refer to the fermented mass resulting from the storage of flowers under anaerobic conditions. In a preferred embodiment, silaged flowers are obtained by treating marigold flowers with at least one enzyme selected from cellulases, hemicellulases, cellobiohydrases, pectinases, galactomannanases, proteases, lipases, or combination thereof, preferably for a period of at least 5 days or about 5 to 10 days. Alternatively, in other embodiments, silaging may be performed without the use of enzymes. The silaged flowers may then be de-juiced and shredded to obtain the shredded marigold flowers. In an embodiment, fresh marigold flowers are silaged, de-juiced and shredded to obtain shredded marigold flowers.

The term “marigold meal”, as used herein, refers to a dehydrated biomass derived from marigold flowers. The term includes meal in any form including, but not limited to, pellet, granule, or powder form.

In an embodiment, the marigold meal is obtained by silaging marigold flowers with at least one enzyme selected from cellulases, hemicellulases, cellobiohydrases, pectinases, galactomannanases, proteases, lipases, and combination thereof, preferably for a period of at least 5 days or about 5 to 10 days; and dehydrating to obtain marigold meal. Preferably, the silaged flowers are further subjected to de-juicing for removal of water content and shredding/grinding, followed by drying under controlled conditions, such that the water content of the marigold meal is in the range of 8 to 12 wt %. Preferably, marigold meal is in a powder form. Accordingly, in an embodiment, the marigold meal is obtained by silaging marigold flowers with at least one enzyme selected from cellulases, hemicellulases, cellobiohydrases, pectinases, galactomannanases, proteases, lipases, and combination thereof, preferably for a period of at least 5 days or about 5 to 10 days; de-juicing; shredding; and dehydrating to obtain the marigold meal. Marigold meal may be further processed to other forms, such as pellets to obtain marigold pellets that are suitable for long term storage and high throughput processing. Shredding may be performed by methods generally known in the art using for example: mechanical shredder, compressor, etc.

In an embodiment, the marigold meal is obtained by a process comprising silaging marigold flowers with at least one enzyme selected from cellulases, hemicellulases, cellobiohydrases, pectinases, galactomannanases, proteases, lipases, and combination thereof, preferably for a period of at least 5 days or about 5 to 10 days; and dehydrating to obtain marigold meal.

In another embodiment, the marigold meal is obtained by a process comprising silaging marigold flowers with at least one enzyme selected from cellulases, hemicellulases, cellobiohydrases, pectinases, galactomannanases, proteases, lipases, and combination thereof, preferably for a period of at least 5 days or about 5 to 10 days; de-juicing; shredding; and dehydrating to obtain marigold meal.

In an embodiment, the shredded marigold flowers are obtained by a process comprising silaging marigold flowers using at least one enzyme selected from cellulases, hemicellulases, cellobiohydrases, pectinases, galactomannanases, proteases, lipases, and combination thereof, preferably for a period of at least 5 days or about 5 to 10 days; de-juicing; and shredding to obtain shredded marigold flowers.

In an embodiment, the method for preparing lutein ester comprises: (a) providing a raw material selected from shredded marigold flowers, marigold meal, or combination thereof; (b) subjecting the raw material to extraction with at least one polar solvent selected from ethanol, methanol, or combination thereof, to obtain a polar solvent fraction and residual biomass; (c) subjecting the residual biomass to extraction with an extractant comprising acetone, to obtain an acetone fraction; and (d) concentrating the acetone fraction.

In an embodiment, the method for preparing lutein ester comprises: (a) providing a raw material selected from shredded marigold flowers, marigold meal, or combination thereof; (b) subjecting the raw material to extraction with at least one polar solvent selected from ethanol, methanol, or combination thereof, to obtain a polar solvent fraction and residual biomass; (c) subjecting the residual biomass to extraction with an extractant comprising acetone, to obtain an acetone fraction; and (d) concentrating the acetone fraction to obtain an extract comprising lutein ester, followed by crystallisation to obtain lutein ester crystals.

In an embodiment, the method for preparing lutein ester, comprises: (a) providing a raw material selected from shredded marigold flowers, marigold meal, or combination thereof; (b) subjecting the raw material to extraction with at least one polar solvent selected from ethanol, methanol, or combination thereof, to obtain a polar solvent fraction and residual biomass; (c) subjecting the residual biomass to extraction with an extractant comprising acetone, to obtain an acetone fraction comprising xanthophylls; and (d) concentrating the acetone fraction to obtain an extract comprising lutein ester, and subjecting the extract to crystallisation to obtain lutein ester crystals.

The step of subjecting the raw material to extraction, in the method according to embodiments herein, comprises extracting the raw material using a polar solvent.

The term “extraction” or “extracting”, as used herein, refers to solvent extraction that achieves transference of substances from one phase to another. The phase may be solid or liquid, accordingly, the extraction may be liquid-liquid extraction or solid-liquid extraction. The extraction process may be performed one or more times. The extraction may be performed in a solvent extractor. In an embodiment, the extraction step is repeated 1 to 5 times using fresh solvent each time. In an embodiment, the extraction step is repeated 1, 2, 3, 4, 5 or more times, preferably 2 to 5 times, using fresh solvent.

In an embodiment, the raw material is subjected to extraction with at least one polar solvent, preferably selected from ethanol, methanol, or combination thereof.

In an embodiment, the ratio of the raw material and polar solvent is in the range of 1:2 to 1:8, or 1:3 to 1:6, preferably 1:4, 1:5, or 1:6. In an embodiment, the ratio of the shredded marigold flowers and polar solvent is in the range of 1:2 to 1:8, or 1:3 to 1:6, preferably 1:4, 1:5, or 1:6. In an embodiment, the ratio of the marigold meal and polar solvent is in the range of 1:2 to 1:8, or 1:3 to 1:6, preferably 1:4, 1:5, or 1:6.

In an embodiment, the extraction using a polar solvent is performed at a temperature in the range of 30 to 60° C., preferably 40 to 50° C., with continuous circulation of the solvent for a period of 20 to 100 minutes, preferably 20 to 60 minutes or 20, to 40 minutes, more preferably 30 minutes.

In another embodiment, the extraction using polar solvent is repeated two to five times to obtain multiple fractions, which can then be combined. The extraction using polar solvent, in the method according to embodiments herein, achieves a polar solvent fraction and a residual biomass. The term “polar solvent fraction” or “polar solvent miscella”, as used herein, is a fraction of solvent mixture that leaves a solvent extractor. In an embodiment, the polar solvent fraction is obtained after the extraction. According to embodiments herein, the polar solvent is an ethanol fraction comprising polyphenols, such as quercetagetin. In an embodiment, polar solvent fraction comprises at least one polyphenol selected from quercetagetin, 6-hydroxy kaempferol, quercetin, patuletin, or mixtures thereof.

The term “residual biomass”, as used herein, refers to the solid mass obtained after removal of the polar solvent fraction. In an embodiment, the residual biomass is obtained after the extraction as a residue.

In an embodiment, the residual biomass is subjected to extraction with an extractant comprising acetone to obtain an acetone fraction. In an embodiment, the acetone fraction comprises xanthophylls. In an embodiment, the acetone fraction comprises xanthophylls including lutein ester, and zeaxanthin ester. Extraction using the extractant comprising acetone may be repeated, preferably 1 to 5 times, to achieve maximum transference for the xanthophyll from the residual biomass to the acetone fraction. In an embodiment, the extraction using extractant comprising acetone may be performed 1 to 5 times, or 1 to 4 times, preferably 4 times using fresh extractant each time. In an embodiment, the extractant is acetone or acetone solution.

In an embodiment, the ratio of said residual biomass and extractant is in the range of 1:2 to 1:8, preferably 1:5.

In an embodiment, there is provided a method wherein subjecting the residual biomass to extraction with an extractant is performed at a temperature in the range of 25 to 52° C., preferably 30 to 45° C. or 40 to 45° C., with continuous circulation of the extractant for a period of 20 to 60 minutes, preferably 20 to 40 minutes, more preferably 30 minutes. In another embodiment, there is provided a method wherein subjecting the residual biomass to extraction with the extractant is performed at a temperature in the range of 40 to 45° C., with continuous circulation of the extractant comprising acetone for a period of 20 to 40 minutes, preferably 30 minutes.

In another embodiment, the extraction using acetone is repeated two to five times to obtain multiple fractions which can then be combined. The extraction using acetone, in the method according to embodiments herein, achieves an acetone fraction. The term “acetone fraction” or “acetone miscella”, as used herein, refers to a fraction having acetone that leaves the extractor and comprises xanthophylls.

The acetone fraction, according to embodiments herein, is further concentrated. In an embodiment, there is provided a method comprises concentrating the acetone fraction. In another embodiment, there is provided a method comprising concentrating the acetone fraction to obtain an extract or an extract comprising lutein ester. The acetone fraction may be concentrated to remove the solvent completely (i.e. desolventized), or until the extract comprises 1 to 12 wt %, 1 to 10 wt %, 1 to 5 wt %, 5 to 12 wt %, or 5 to 10 wt % extractives.

In another embodiment, the method comprises concentrating the acetone fraction, wherein the concentrating is performed to obtain an extract comprising 1 to 12 wt %, 1 to 10 wt %, 1 to 5 wt %, 5 to 12 wt %, or 5 to 10 wt % extractives.

In an embodiment, the method comprises concentrating the acetone fraction, wherein the concentrating is performed to obtain an extract comprising 1 to 12 wt % extractives, preferably 1 to 5% extractives when the raw material is shredded marigold flowers, preferably 5 to 10% extractives when the raw material is marigold meal.

In an embodiment, the acetone fraction is concentrated until the extract comprises 1 to 12 wt %, 1 to 10 wt %, 1 to 5 wt %, 5 to 12 wt %, or 5 to 10 wt % extractives.

In an embodiment, the method comprises concentrating the acetone fraction to remove the solvent (i.e. acetone) or for desolventizing, and to obtain an extract. In an embodiment, the extract obtained after desolventizing or concentrating the acetone fraction to remove the solvent, is an oleoresin. In an embodiment, the method comprises concentrating the acetone fraction, wherein said concentrating is performed to obtain an extract comprises xanthophyll in the weight range of 15 to 30 wt %, lutein in the weight range of 70 to 80 wt %, and zeaxanthin in the weight range of 3 to 6 wt %. In an embodiment, the extract is an oleoresin comprising xanthophyll in the weight range of 15 to 30 wt %, lutein in the weight range of 70 to 80 wt %, and zeaxanthin in the weight range of 3 to 6 wt %, wherein the yield of oleoresin is in the range of 5 to 10 wt %, or 6 to 10 wt %, in respect of the raw material.

Concentration may be performed by methods well known in the art. For example, the extract may be concentrated using a vacuum evaporator. Extractives, according to the present disclosure, refer to the components that are transferred into the acetone phase from the residual biomass. The acetone fraction is concentrated using a method known in the field to obtain an extract enriched with the extractives.

In an embodiment, the extract comprises 1 to 12% extractives. In another embodiment, the extract comprises 5 to 12% or 5 to 10% extractives. In another embodiment, the extract comprises 1 to 5% extractives. In another embodiment, the extract comprises 8 to 10% extractives.

In an embodiment, the extract comprises 1 to 5% extractives when the raw material is shredded marigold flowers. In an embodiment, the extract comprises 5 to 10% extractives when the raw material is marigold meal.

In an embodiment, the method comprises subjecting said extract to crystallization to obtain crystals comprising lutein ester, and mother liquor. In an embodiment, the crystallization is performed to obtain crystals comprising lutein ester, and mother liquor.

In an embodiment, crystallization is performed at a temperature in the range of 7 to 40° C., for a period of 8 to 60 hours, 8 to 50 hours, 10 to 50 hours, 10 to 40 hours, 10 to 30 hours, 22 to 50 hours, or 20 to 50 hours, preferably selected from 10 hours, 12 hours, 48 hours, or 60 hours. In an embodiment, crystallization is performed at a temperature in the range of 7 to 40° C., 9 to 37° C., 10 to 37° C., 20 to 37° C., 7 to 20° C., or 30 to 37° C.

In another embodiment, crystallization is performed at a temperature in the range of 9 to 37° C., for a period of 20 to 50 hours, preferably 48 hours. In one another embodiment, crystallization is performed at a temperature in the range of 20 to 35° C., for a period of 20 to 50 hours, preferably 48 hours. In yet another embodiment, crystallization is performed in a continuous stirred tank reactor (CSTR) at a temperature in the range of 30 to 37° C., for a period of 22 to 50 hours, preferably 48 hours.

In an embodiment, the method comprises: (a) providing a raw material selected from shredded marigold flowers, marigold meal, or combination thereof, wherein the raw material is marigold meal; (b) subjecting the raw material to extraction with at least one polar solvent selected from ethanol, methanol, or combination thereof, to obtain a polar solvent fraction and residual biomass; (c) subjecting the residual biomass to extraction with an extractant comprising acetone, to obtain an acetone fraction; and (d) concentrating the acetone fraction to obtain an extract comprising lutein ester, followed by crystallisation to obtain lutein ester crystals, wherein the crystallization is performed at a temperature in the range of 7 to 40° C., preferably 10 to 37° C. or 20 to 37° C., for a period of 20 to 50 hours, preferably 48 hours.

In an embodiment, the method comprises: (a) providing a raw material selected from shredded marigold flowers, marigold meal, or combination thereof, wherein the raw material is shredded marigold flowers; (b) subjecting the raw material to extraction with at least one polar solvent selected from ethanol, methanol, or combination thereof, to obtain a polar solvent fraction and residual biomass; (c) subjecting the residual biomass to extraction with an extractant comprising acetone, to obtain an acetone fraction; and (d) concentrating the acetone fraction to obtain an extract comprising lutein ester, and subjecting the extract to crystallisation to obtain lutein ester crystals, wherein the crystallization is performed at a temperature in the range of 7 to 40° C., preferably 10 to 37° C., for a period of 10 to 50 hours or 20 to 50 hours, preferably 10, 12 or 24 hours. In an embodiment, there is provided a method, wherein the extraction step of step (b) and/or step (c) is repeated at least once, preferably 2 to 5 times, preferably to achieve complete extraction.

The lutein ester crystals, according to embodiments herein, are further subjected to filtration and drying to obtain lutein ester crystals. Filtration and drying may be performed using methods generally known in the art. Drying, preferably is performed using a de-humidified air dryer (DHAD) at a temperature in a range of 25 to 50° C., 45 to 48° C., or 45 to 48° C. In an embodiment, there is provided a method, wherein the method comprises filtrating and drying to obtain lutein ester crystals, wherein drying in performed at a temperature in a range of 25 to 50° C., 45 to 48° C., or 45 to 48° C.

In an embodiment, the yield of said lutein ester is in the range of 0.25 to 2.5 wt %, in respect of the raw material. In an embodiment, the yield of said lutein ester is in the range of 1.1 to 2.5 wt %, in respect of the marigold meal. In another embodiment, the yield of said lutein ester is in the range of 2.1 to 2.5 wt %, in respect of the marigold meal. In an embodiment, the yield of said lutein ester is in the range of 0.25 to 1.0 wt %, in respect of the shredded marigold flowers.

In an embodiment, the yield of said lutein ester crystals is in the range of 0.25 to 2.5 wt %, in respect of the raw material, preferably 1.1 to 2.5 wt % when the raw material is marigold meal, or preferably 0.25 to 1.0 wt % when the raw material is shredded marigold flowers.

In an embodiment, the purity of lutein ester is at least 55 wt %. In an embodiment, the purity of lutein ester is at least 55 wt %, at least 58 wt %, at least 60 wt %, at least 62 wt %, at least 64 wt %, at least 66 wt %, at least 68 wt %, at least 70 wt %, at least 72 wt %, at least 74 wt %, at least 76 wt %, at least 78 wt, at least 80 wt %, at least 82 wt %, at least 84 wt %, at least 86 wt %, at least 87 wt %, or at least 88 wt %. In an embodiment, the purity of lutein ester is in the range of 55 to 62 wt %. In another embodiment, the purity of lutein ester is in the range of 70 to 88 wt % or 75 to 88 wt %. In yet another embodiment, there is provided a method wherein the raw material is marigold meal, and the purity of lutein ester is in the range of 84 to 88 wt %. In yet another embodiment, there is provided a method wherein the raw material is marigold meal, and the purity of lutein ester is in the range of 75 to 88 Wt %.

In an embodiment, the purity of lutein ester is in the range of 70 to 88 wt %, and wherein said crystals further comprises zeaxanthin ester in the range of 2 to 6 wt %. In another embodiment, the purity of lutein ester is in the range of 84 to 88 wt %, and wherein said crystals further comprises zeaxanthin ester in the range of 3 to 6 wt %.

The mother liquor, according to embodiments herein, is further concentrated to obtain an oleoresin. In an embodiment, the oleoresin is subjected to saponification to obtain free lutein with at least 74% purity. The term “saponification”, as used herein, refers to the hydrolysis of ester, for eg: lutein ester into free lutein and potassium salt of fatty acids. Saponification may be carried out in the presence of an alkali.

In an embodiment, the method further comprises concentrating said mother liquor to obtain an oleoresin, wherein the oleoresin comprises xanthophyll in an amount of at least 12%, or 12 to 16 wt %, of which lutein is in the range of 70 to 75 wt % and zeaxanthin is in the range of 3 to 6 wt %.

The polar solvent fraction obtained by the method, according to embodiments herein, may further be purified with carbon in ethanol. In an embodiment, the method further comprises subjecting the polar solvent fraction to purification with carbon in ethanol to obtain quercetagetin. In an embodiment, there is provided a method, wherein said quercetagetin is of at least 90% purity.

Embodiments herein achieve lutein ester crystals of high purity, and reduced or no impurities. The term impurities, as used herein, include at least one ingredient selected from protein, ash, pesticides, poly aromatic hydrocarbons (PAH), benzopyrene, pesticides, furans, dioxins, melamine, ethylene oxide, and nitrosamine. In an embodiment, there is provided a method, wherein said crystals further comprises protein at a weight range of 0 to 0.5%, ash at a weight range of 0 to 0.2%; poly aromatic hydrocarbons (PAH) in an amount of 0 to 2 ppb, benzopyrene in an amount of 0 to 0.5 ppb, pesticides in an amount of 0 to 0.01 ppm, dioxins in an amount of 0 to 0.5 pg/g, melamine in an amount of 0 to 0.1 mg/kg, ethylene oxide in an amount of 0 to 10 ppb, and nitrosamine in an amount of 0 to 0.01 mg/kg.

In an embodiment, there is provided a method, wherein said crystals have an impurity profile comprising 0 to 0.5% of protein, 0 to 0.2% of ash; 0 to 2 ppb of poly aromatic hydrocarbons (PAH), 0 to 0.5 ppb of benzopyrene, 0 to 0.01 ppm of pesticides, 0 to 0.5 pg/g of dioxins, 0 to 0.1 mg/kg of melamine, 0 to 10 ppb of ethylene oxide, and 0 to 0.01 mg/kg of nitrosamine.

Embodiments herein further provide a method for preparing lutein ester using an extractant comprising isopropanol. In an embodiment, the method comprises: (a) providing a raw material selected from shredded marigold flowers, marigold meal, or combination thereof, (b) subjecting the raw material to extraction with an extractant comprising isopropanol, to obtain an isopropanol fraction; and (c) concentrating the isopropanol fraction and optionally followed by crystallization to obtain lutein ester crystals.

In an embodiment, the method comprises: (a) providing a raw material selected from shredded marigold flowers, marigold meal, or combination thereof; (b) subjecting the raw material to extraction with an extractant comprising isopropanol, to obtain an isopropanol fraction; and (c) concentrating the isopropanol fraction to obtain an extract followed by crystallization to obtain lutein ester crystals.

In an embodiment, the method comprises: (a) providing a raw material selected from shredded marigold flowers, marigold meal, or combination thereof, (b) subjecting the raw material to extraction with an extractant comprising isopropanol, to obtain an isopropanol fraction; and (c) concentrating the isopropanol fraction followed by crystallization. In an embodiment, there is provided a method, wherein the extraction step of step (b) is repeated at least once, preferably 2 to 5 times, preferably to achieve complete extraction.

In an embodiment, ratio of the raw material and extractant is in the range of 1:2 to 1:8, preferably 1:5. In an embodiment, ratio of the shredded marigold flowers and extractant is in the range of 1:2 to 1:8, preferably 1:5. In an embodiment, ratio of the marigold meal and extractant is in the range of 1:2 to 1:8, preferably 1:5.

In an embodiment, the raw material is extracted using an extractant comprising isopropanol at a temperature in the range of 50 to 60° C., 53 to 60° C., 53 to 58° C., or 54 to 58° C., preferably 55° C., with continuous circulation of the extractant for a period of 20 to 40 minutes, preferably 30 minutes.

In another embodiment, the extraction using extractant is repeated two to five times to obtain multiple fractions which can then be combined. The extraction using extractant comprising isopropanol, in the method according to embodiments herein, achieves an isopropanol fraction. The term “isopropanol fraction” or “isopropanol miscella”, as used herein, is a fraction of isopropanol solvent mixture that leaves a solvent extractor. In an embodiment, the isopropanol fraction is obtained after the extraction. The isopropanol fraction, according to embodiments herein, is further concentrated to obtain an extract comprising 1 to 12 wt %, 5 to 12% or 5 to 10 wt % extractives, using known methods. For example, the isopropanol fraction may be concentrated using a continuous vacuum evaporator to obtain an extract.

In an embodiment, the extract comprises 1 to 12%, 5 to 12% or 5 to 10% extractives. In another embodiment, the extract comprises 8 to 10% extractives.

In an embodiment, the method comprises concentrating the isopropanol fraction, wherein said concentrating is performed to obtain an extract comprising 1 to 12%, 5 to 12%, 5 to 10%, or 8 to 10% extractives. The isopropanol fraction may, alternatively, be concentrated to desolventize.

In an embodiment, the method further comprises subjecting said extract to crystallization to obtain crystals comprising lutein ester, and mother liquor. In an embodiment, the crystallization is performed to obtain crystals comprising lutein ester, and mother liquor.

In an embodiment, the crystallization is performed at a temperature in the range of 10 to 55° C., 10 to 45° C., 20 to 40° C., 30 to 40° C., for a period of 5 to 90 hours, 20 to 80 hours, 20 to 75 hours, preferably 48, 72, or 24 hours, preferably 48 hours. In another embodiment, crystallization is performed at a temperature in the range of 30 to 40° C., for a period of 5 to 90 hours, 20 to 80 hours, 20 to 75 hours, preferably 48, 72, or 24 hours. In one another embodiment, crystallization is performed at a temperature in the range of 20 to 35° C., for a period of 24 to 72 hours, preferably 48 hours. In yet another embodiment, crystallization is performed at a temperature in the range of 33 to 35° C., for a period of 24 to 72 hours, preferably 48 hours.

In an embodiment, the yield of said lutein ester is in the range of 2 to 3 wt %, in respect of the marigold meal. In another embodiment, the yield of said lutein ester is in the range of 2.5 to 3 wt %, in respect of the marigold meal.

In an embodiment, the purity of lutein ester is at least 55 wt %. In an embodiment the purity of lutein ester is at least 55 wt %, at least 58 wt %, at least 60 wt %, at least 62 wt %, at least 64 wt %, at least 66 wt %, at least 68 wt %, at least 70 wt %, at least 72 wt %, at least 74 wt %, at least 76 wt %, or at least 87 wt. In an embodiment, the purity of lutein ester is in the range of 55 to 62 wt %. In an embodiment, the purity of lutein ester is in the range of 70 to 75 wt %, and wherein said crystals further comprise zeaxanthin ester in the range of 2 to 6 wt %. In another embodiment, the purity of lutein ester is in the range of 72 to 75 wt %, and wherein said crystals further comprise zeaxanthin ester in the range of 3 to 6 wt %.

In an embodiment, the method further comprises concentrating said mother liquor to obtain an oleoresin, wherein the oleoresin comprises xanthophyll in the range of 5 to 8 wt %, of which lutein is in the range of 70 to 75 wt % and zeaxanthin is in the range of 3 to 6 wt %.

Although the subject matter has been described in considerable detail with reference to certain examples and implementations thereof, other implementations are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar to or equivalent to those described herein can be used in the practice of the disclosed methods and compositions, the exemplary methods, devices, and materials are described herein. It is to be understood that this disclosure is not limited to particular methods, and experimental conditions described, as such methods and conditions may apply.

### Materials

Marigold flowers were obtained from plants grown from seeds procured from Thailand.

Solvents used in the present disclosure, such as ethanol was procured from Godavari biorefineries Ltd, Karnataka, India; acetone was procured from Hindustan Organic Chemicals Ltd., Cochin, India; and isopropyl alcohol was procured from Karnataka Chemical Industries, Bengaluru, India.

### Example 1: Preparation of Marigold Meal for Lutein Ester Extraction

### Enzyme Assisted Silaging

Fresh marigold flowers with xanthophyll content of 0.04 to 0.3%; polyphenols and waxes content of 8 to 11% and moisture content of 88 to 92% by weight were mixed with an optimal mix of enzymes, such as cellulases, hemicellulases, cellobiohydrases, pectinases, galactomannanases, proteases and lipases. The dosage of enzyme mixture ranges from 0.15% of fresh flower weight. The fresh flower biomass was simultaneously ensiled and hydrolyzed for a period of about 8 days, to obtain an enzyme-silaged biomass.

The enzyme-silaged biomass was de-juiced to remove water content and then shredded, and dried to obtain the marigold meal. The marigold meal comprised xanthophylls in a range of 1.8 to 3.0% in respect of the marigold meal as determined by UV analysis; and lutein in a range of 75 to 84% in respect of the xanthophyll content (Table 1), as determined by High Performance Liquid Chromatography (HPLC) analysis.

TABLE 1

| Marigold Meal components | Amount in wt % |
|---|---|
| Xanthophylls | 1.8 to 3.0% |
| Lutein | 1.3 to 2.5% |
| Zeaxanthin | 0.1 to 0.14% |
| Polyphenols | 3 to 5% |

### Example 2: Extract Preparation from Marigold Meal

250 g marigold meal (prepared using Example 1) was taken in an extractor and treated with ethanol in the weight ratio of marigold meal to ethanol (1:4). The mixture was heated at 45° C. with stirring and extracted with specified volume of ethanol. The mixture was then filtered, and the residue re-extracted with ethanol until the ethanol solubles were completely extracted, 4 cycles of extraction performed. The residual biomass was then treated with acetone in the weight ratio of residual biomass to acetone (1:5) at 40° C. Repeated extraction was done with acetone to recover all the xanthophylls from the biomass. The acetone miscella was then concentrated to desolventize and yield an extract. The extract was tested for xanthophyll, lutein and zeaxanthin content. Table 1.1 illustrates the yield, xanthophyll content, lutein content and zeaxanthin content of the extract.

TABLE 1.1

| | Yield (%) of | | | |
|---|---|---|---|---|
| Experiment | Extract | Xanthophyll % | Lutein % | Zeaxanthin % |
| 1 | 8.4 | 22 | 76.2 | 4.6 |
| 2 | 8.6 | 21.3 | 75.8 | 4.8 |

Extraction of Lutein Ester from Marigold Meal Using Ethanol-Acetone

Marigold meal (250 g) (prepared using Example 1 of the present disclosure) was taken in a 5 L Erlenmeyer flask and was treated with ethanol (polar solvent) in the weight ratio of marigold meal:ethanol ranging from 1:2 to 1:8, as shown in Table 1.2, Experiments 1 to 3. The mixture was heated at 45° C. while stirring and with continuous ethanol (polar solvent) circulation for a period of 30 minutes. The mixture was then filtered, and the residue was re-extracted with ethanol until the ethanol (polar solvent) soluble content, such as polyphenols were completely extracted, preferably 4 cycles of extraction were performed. The ethanol (polar solvent) fraction was then filtered, and ethanol was evaporated. This ethanol (polar solvent) fraction was then evaporated, subjected to carbon treatment and crystallization to obtain quercetagetin, the major flavonoid (polyphenol) present in marigold flowers. The polyphenol content was determined using UV analysis and quercetagetin content was determined using HPLC analysis. The yield of polyphenol was calculated using the following formula:

$$Yield of Polyphenol = \frac{Wt . of polyphenol fraction after distilling out solvent}{Wt . of meal taken for extraction} \times 100$$

The residual biomass was extracted with acetone in the weight ratio of residual biomass:acetone ranging from 1:2 to 1:8, as shown in Table 1.2. The mixture was preferably heated to a temperature in the range of 45° C., with continuous circulation of acetone for a period of 30 minutes. Repeated extraction was performed with acetone to recover all the xanthophylls from the meal. The acetone miscella was then concentrated to obtain an extract, concentration was performed until 5 to 10% of extractives was present in the extract. The extract comprises lutein ester as shown in Table 1.2. The extract was then subjected to crystallization at 35° C. for 24 to 72 hours, preferably 48 hours, as shown in Table 1.2, Experiments 4 to 8. The crystals of trans lutein esters were settled down, filtered, and dried in dehumidified air dryer (DHAD) at 46° C. The total carotenoids were estimated by UV-Vis spectrophotometer and lutein ester contents were measured by HPLC in normal phase after saponification using methanolic KOH at 56° C. for 1 hour. HPLC was carried out using hexane/ethyl acetate (65:35) as mobile phase; column; Phenomenex Luna C18 column at a wavelength of 446 nm. The yield of lutein ester was calculated using the following formula:

$$Yield of lutein ester = \frac{Wt . of lutein ester crystals}{Wt . of meal taken for extraction} \times 100$$

Similarly, the ethanol-acetone extraction may also be adopted for the marigold flowers at wet stage, silaged flowers without reducing moisture (avoiding drying). The use of alcohols like ethanol can dehydrate the wet flowers along with extracting water soluble and polyphenols. This alcohol treated biomass can be used for acetone extraction and crystallization of lutein ester. Table 1.2 depicts the working and non-working examples, wherein experiments 1 to 9 achieved satisfactory results. However, in experiments 10, 11 and 12, no lutein crystals and oleoresin nature was observed.

### Observation and Results

The extraction method using ethanol and acetone yielded lutein ester crystals in the range of 1.1 to 2.5% of the meal with a purity of trans lutein ester in the isolated crystals of 84 to 88% and 2 to 6% trans zeaxanthin ester (Table 1.2). Particularly, Experiment No. 6, which involved the use of 1:4, i.e. meal: ethanol, weight ratio and 1:5, i.e. meal: acetone, weight ratio and with crystallization carried out at 35° C. and 48 hours yielded 87% lutein ester crystals.

The ethanol (polar solvent) fraction yielded 22 wt % of polyphenol products, comprising quercetagetin of 200 purity, which was further purified to obtain quercetagetin with more than 900 purity (Table 1.3). The by-product obtained by desolventising (removal of acetone) the mother liquor showed quality similar to conventional (hexane extracted) oleoresin. Yield of the by-product formed was 4 to 6% oleoresin in respect of the meal, comprising 12 to 16 wt % of xanthophylls. The xanthophylls comprised of 70 to 7500 trans lutein in respect of the xanthophylls as shown in Table 1.4. This oleoresin was saponified to get free lutein with >7400 purity.

TABLE 1.2
Experimental conditions for obtaining lutein ester using ethanol-acetone extraction method.

| | | | | Crystallization | | | | | |
|---|---|---|---|---|---|---|---|---|---|
| | | | | condition | | % | | | |
| | Meal | Meal | % Total | | Temp | Yield | | Lutein | Zea |
| | to | to | Extractives | Time | in | of | | Ester | esters |
| | Ethanol | Acetone | for | in | deg | Lutein | % Total | Purity | Purity |
| Experiment | ratio | ratio | crystallization | hours | C. | Ester | Carotenoids | (%) | (%) |
| 1 | 1:2 | 1:2 | 10 | 48 | 35 | 2.2 | 92 | 85.5 | 3.2 |
| 2 | 1:4 | 1:5 | 10 | 48 | 35 | 2.4 | 93 | 86.5 | 3.3 |
| 3 | 1:8 | 1:8 | 10 | 48 | 35 | 2:3 | 91 | 84.6 | 3.0 |
| 4 | 1:4 | 1:5 | 5 | 48 | 35 | 2:2 | 91 | 85 | 2.9 |
| 5 | 1:4 | 1:5 | 8 | 48 | 35 | 2 | 92 | 86 | 3.3 |
| 6 | 1:4 | 1:5 | 10 | 48 | 35 | 2.4 | 93 | 87 | 3.0 |
| 7 | 1:4 | 1:5 | 10 | 24 | 35 | 2 | 90 | 84 | 3.1 |
| 8 | 1:4 | 1:5 | 10 | 48 | 35 | 2.4 | 93 | 87 | 3.3 |
| 9 | 1:4 | 1:5 | 10 | 72 | 35 | 2.4 | 91 | 85 | 3.0 |

| | | | | | | |
|---|---|---|---|---|---|---|
| 10 | 1:10 | 1:10 | 10 | 48 | 35 | No crystals, oleoresin Nature |
| 11 | 1:4 | 1:5 | 20 | 48 | 35 | No crystals, oleoresin Nature |
| 12 | 1:4 | 1:5 | 10 | 1 | 35 | No crystals, oleoresin Nature |

TABLE 1.3
Ethanol (polar solvent) fraction

| % Yield of Ethanol Fraction | Polyphenol % | Quercetagetin % |
|---|---|---|
| 22 | 30 | 20 |

TABLE 1.4
Products from mother liquor

| Oleoresin Yield % | Xanthophyll % | Lutein % | Zeaxanthin % |
|---|---|---|---|
| 6 | 15 | 75 | 4.8 |

### Example 2.1: Large Scale Preparation of High Purity Crystalline Lutein Ester from Marigold Meal Using Ethanol-Acetone Extraction Method

100 kg marigold meal was taken in an extractor and treated with ethanol in the weight ratio of marigold meal to ethanol (1:4). The mixture was heated at 45° C. with stirring and extracted with specified volume of ethanol. The mixture was then filtered, and the residue re-extracted with ethanol until the ethanol solubles were completely extracted, 4 cycles of extraction performed. The residual biomass was then treated with acetone in the weight ratio of residual biomass to acetone (1:5) at 40° C. Repeated extraction was done with acetone to recover all the xanthophylls from the biomass. The acetone miscella concentrated to obtain extract with 10% extractives and kept for crystallization at 35° C. for 48 hrs. The crystals of trans lutein esters settled were filtered out, dried under DHAD at 45 to 48° C. The filtered miscella (mother liquor) desolventized to yield oleoresin.

TABLE 2.1.1
Product profile of high purity crystalline
lutein ester from marigold meal

| | % Yield of Lutein | | |
|---|---|---|---|
| Experiment | Ester from Meal | % Total Carotenoids | % Lutein ester |
| 1 | 2.1 | 91 | 85.1 |
| 2 | 2.4 | 92 | 86 |
| 3 | 2.2 | 91 | 85.5 |

Impurities (contaminants) generally seen in marigold products are polycyclic aromatic hydrocarbons, dioxins, nitrosamine, ethylene oxide etc.

TABLE 2.1.2
Impurity profiling of high purity crystalline lutein ester

| Parameters | Experiment 1 | Experiment 2 | Experiment 3 |
|---|---|---|---|
| Protein % | 0.4 | 0.41 | <0.1 |
| Ash % | <0.1 | <0.1 | <0.1 |
| PAH(poly aromatic | Not detected | Not detected | Not detected |
| hydrocarbons) in ug/kg | | | |
| Benzopyrene in ug/kg | Not detected | Not detected | Not detected |
| Pesticides | Not detected | Not detected | Not detected |
| Dioxins, furans in pg/g | 0.495 | 0.451 | 0.477 |
| Melamine in mg/kg | <0.1 | <0.1 | <0.1 |
| Ethylene Oxide in ppb | <10 | <10 | <10 |
| Nitrosamine in mg/kg | <0.01 | <0.01 | <0.01 |

Ethanol Extract (i.e. Polar Solvent Fraction)

Ethanol miscella from the experiment filtered and evaporated to an extract with polyphenols as the active ingredients, quercetagetin being the major polyphenol.

TABLE 2.1.3
Ethanol (polar solvent) fraction

| | % Yield of | | |
|---|---|---|---|
| | Ethanol Extract | % Polyphenol | % Quercetagetin |
| Experiment 1 | 20 | 30 | 21 |
| Experiment 2 | 21 | 32 | 22 |
| Experiment 3 | 18 | 31 | 20 |

Isolation of Quercetagetin from the Ethanol Extract (i.e. Polar Solvent Fraction)

The ethanol extract on purification with carbon in ethanol medium yielded Quercetagetin >90% purity.

TABLE 2.1.4
Isolation of Quercetagetin

| | % Yield | % Polyphenols | % Quercetagetin |
|---|---|---|---|
| Experiment 1 | 3 | 92 | 91 |
| Experiment 2 | 3.3 | 91 | 90.8 |
| Experiment 3 | 3.5 | 92 | 91.2 |

Marigold Oleoresin from the Mother Liquor after Crystallising Lutein Ester

The by-product obtained by desolventising the mother liquor is similar in quality to conventional (hexane extracted) oleoresin as shown in Table 2.1.5. This oleoresin was saponified to get free lutein with minimum 74% purity.

TABLE 2.1.5
Marigold Oleoresin from Lutein Ester By-product

| | % Yield | | | % |
|---|---|---|---|---|
| | of | % | % Lutein | Zeaxanthin |
| | Oleoresin | Xanthophylls | by HPLC | by HPLC |
| Experiment 1 | 5 | 14 | 75 | 4.2 |
| Experiment 2 | 4.8 | 15.2 | 73 | 4.6 |
| Experiment 3 | 5.1 | 14.3 | 72 | 4.1 |

### Example 2.2: Large Scale Preparation of High Purity Crystalline Lutein Ester from Marigold Meal Using Ethanol-Acetone

100 kg Marigold meal was taken in an extractor and treated with ethanol in the weight ratio of marigold meal to ethanol (1:4). The mixture was heated at 40 to 50° C. with stirring and extracted with specified volume of ethanol. The mixture was then filtered, and the residue re-extracted with ethanol until the ethanol solubles are completely extracted, 4 cycles of extraction were performed. The residual biomass was treated with acetone in the weight ratio of residual biomass to acetone (1:5) at 40 to 45° C. Repeated extraction done with acetone to recover all the xanthophylls from the biomass. The acetone miscella concentrated to 10% concentration and kept for crystallization at 25° C. for 60 hrs. The crystals of trans lutein esters settled were filtered out, dried under DHAD at 45 to 48° C.

The filtered miscella (mother liquor) desolventized to obtain oleoresin marigold.

TABLE 2.2.1
Product profile of crystalline lutein ester from marigold meal

| | Product Yield % | | |
|---|---|---|---|
| Experiment | from meal | Total Carotenoid % | Lutein ester % |
| 1 | 3.0 | 62 | 58 |
| 2 | 3.2 | 64 | 59.8 |
| 3 | 2.9 | 66 | 61.7 |

TABLE 2.2.2
Impurity profiling of crystalline lutein ester

| Parameters | Experiment 1 | Experiment 2 | Experiment 3 |
|---|---|---|---|
| Protein % | <0.1 | <0.1 | <0.1 |
| Ash % | <0.1 | <0.1 | <0.1 |
| Poly cyclic aromatic | Not detected | Not detected | Not detected |
| hydrocarbons in ug/kg | | | |
| Benzopyrene in ug/kg | Not detected | Not detected | Not detected |
| Pesticides | Not detected | Not detected | Not detected |
| Dioxins, furans in pg/g | 0.482 | 0.484 | 0.478 |
| Melamine in mg/kg | <0.1 | <0.1 | <0.1 |
| Ethylene Oxide in ppb | <10 | <10 | <10 |
| Nitrosamine in mg/kg | <0.01 | <0.01 | <0.01 |

Polyphenols from the Ethanol Extract (i.e. Polar Solvent Fraction)

Ethanol miscella from the experiments filtered and evaporated to an extract having polyphenols as the actives.

TABLE 2.2.3
Ethanol Extract

| | | % Polyphenol | % Quercetagetin |
|---|---|---|---|
| | % Yield of Ethanol | in the | in the |
| | extract | ethanol extract | ethanol extract |
| Experiment | 24 | 34 | 20 |
| 1 | | | |
| Experiment | 19 | 31 | 24 |
| 2 | | | |
| Experiment | 21 | 35 | 22 |
| 3 | | | |

Isolation of Quercetagetin from the Ethanol Extract

Quercetagetin >90% purity isolated from the ethanol extract by carbon treatment in ethanol medium.

TABLE 2.2.4
Isolation of quercetagetin

| | Yield % | Polyphenol % | Quercetagetin % |
|---|---|---|---|
| Experiment 1 | 2.8 | 93 | 91.4 |
| Experiment 2 | 3.4 | 91.5 | 90.9 |
| Experiment 3 | 3.3 | 92.3 | 91.8 |

Oleoresin from by-Product of Lutein Ester

The by-product obtained by desolventising the mother liquor shows quality similar to conventional (hexane extracted) oleoresin as shown in Table 2.2.5. This oleoresin was saponified to get free lutein with >74% purity.

TABLE 2.2.5
Marigold oleoresin from lutein ester by-product

| | % Yield of | % | % Lutein | % Zeaxanthin |
|---|---|---|---|---|
| | Oleoresin | Xanthophylls | by HPLC | by HPLC |
| Experiment 1 | 6 | 15.3 | 72 | 3.8 |
| Experiment 2 | 5.4 | 15.7 | 74 | 4.1 |
| Experiment 3 | 5.2 | 14.8 | 73 | 4.3 |

### Example 3: Extraction of Lutein Ester from Fresh Shredded Marigold Flowers Using Ethanol-Acetone

### 3.1: Preparation of Shredded Marigold Flowers for Lutein Ester Extraction

Fresh marigold flowers with xanthophyll content of 0.04 to 0.3%; polyphenols and waxes content of 8 to 11% and moisture content of 88 to 92% by weight were mixed with an optimal mix of enzymes, such as cellulases, hemicellulases, cellobiohydrases, pectinases, galactomannanases, proteases and lipases. The dosage of enzyme mixture ranges from 0.02 to 0.5% of fresh flower weight. The fresh flower biomass was simultaneously ensiled and hydrolyzed for a period of about 5 to 10 days, to obtain an enzyme silaged biomass.

The enzyme silaged biomass was de-juiced to remove surface moisture and then, shredded to get the marigold shredded flower. Shredded flower having xanthophylls in the range of 0.5 to 1.2% with 77 to 87% lutein content. Shredded marigold flowers were extracted with polar solvents such as ethanol, methanol in the weight ratio of the shredded marigold flower: solvent ranging from 1:2 to 1:8, preferably 1:5. The mixture was preferably heated to a temperature in the range of 40 to 50° C., with continuous solvent circulation for a period of around 30 minutes. The extraction procedure was repeated to extract fully the water-soluble components and polyphenols from the flowers resulting in ethanol fraction (polar solvent fraction) and residual biomass. The ethanol fraction was then filtered, and the ethanol was evaporated and was then processed to isolate the quercetagetin, the major flavonoid present in marigold flowers. The residual biomass was extracted with acetone in the weight ratio of the residual biomass: acetone ranging from 1:2 to 1:8, preferably 1:4. The mixture was preferably heated to a temperature in the range of 40 to 45° C., with continuous circulation of the solvent for a period of around 30 minutes. The extraction procedure was repeated to completely extract the xanthophylls. The acetone fraction so obtained was concentrated to obtain extract having extractives in the range of 1 to 5% extractives, and kept for crystallization at 8 to 15° C. for 10 to 24 hrs, preferably 12 hrs. Crystallized Lutein esters were filtered and dried in dehumidified air drier at 45 to 48° C. High purity lutein ester crystals yielded in the range of 0.4 to 0.8% of the shredded marigold flower with a purity of trans lutein ester in the isolated crystals 78 to 85% and 2 to 5% trans zeaxanthin ester. The mother liquor was concentrated to obtain oleoresin with a yield of 0.8 to 1.6% in respect of the shredded marigold flowers and xanthophyll content in the oleoresin is 12 to 16%. Trans lutein in this by-product (i.e. oleoresin) is 68 to 74%.

### 3.2: Lab-Scale Production of High Purity Crystalline Lutein Ester from Marigold Shredded Flower

250 g shredded marigold flower taken in a 2 L Erlenmeyer flask and treated with specified amount of ethanol as shown in Table 3.1. The mixture heated at 40 to 50° C. with stirring and extracted with specified volume of ethanol. The mixture was then filtered, and the residual biomass re-extracted with ethanol until the ethanol solubles are completely extracted, preferably 4 cycles of extraction. The residual biomass was then treated with specified amount of acetone as shown in Table 3.1 at 40 to 45° C. Repeated extractions were done with acetone to recover all the xanthophylls from the biomass. The acetone miscella was concentrated to specified level of concentration as shown in Table 3.1 and kept for crystallization for specified time and temperature. The crystals of trans lutein esters settled were filtered out, dried under DHAD at 45 to 48° C. The total carotenoids estimated by UV-Vis and lutein esters measured by HPLC after saponification.

TABLE 3.1
Experimental conditions for High purity Lutein ester from Marigold Shredded Flower

| | Shredded | | | Crystallization | | | | | |
|---|---|---|---|---|---|---|---|---|---|
| | flower | | | condition | | % | | | |
| | (SF) to | SF to | Total | | Temp | Yield | | % | % |
| | Ethanol | Acetone | Extractives | Time | in | of | | Lutein | Zeaxanthin |
| | Ratio | ratio | % kept for | in | deg | Lutein | % Total | esters | esters |
| Experiment | used | used | crystallization | hrs | C. | ester | Carotenoids | Purity | Purity |
| 1 | 1:2 | 1:2 | 2 | 10 | 10 | 0.5 | 84 | 78.9 | 3.1 |
| 2 | 1:5 | 1:4 | 2 | 12 | 10 | 0.65 | 87 | 81.8 | 3.5 |
| 3 | 1:8 | 1:8 | 2 | 24 | 10 | 0.55 | 85 | 79.9 | 3.2 |
| 4 | 1:5 | 1:4 | 1 | 12 | 10 | 0.5 | 85 | 80 | 2.9 |
| 5 | 1:5 | 1:4 | 2 | 12 | 10 | 0.65 | 87 | 81.8 | 3.5 |
| 6 | 1:5 | 1:4 | 5 | 12 | 10 | 0.6 | 86 | 80.8 | 3.1 |
| 7 | 1:5 | 1:4 | 2 | 12 | 8 | 0.6 | 83 | 78 | 3.0 |
| 8 | 1:5 | 1:4 | 2 | 12 | 10 | 0.65 | 87 | 81.8 | 3.5 |
| 9 | 1:5 | 1:4 | 2 | 12 | 15 | 0.52 | 84 | 79 | 3.4 |

| | | | | | | |
|---|---|---|---|---|---|---|
| 10 | 1:10 | 1:10 | 2 | 12 | 10 | No Crystals, Oleoresin nature |
| 11 | 1:5 | 1:4 | 20 | 12 | 10 | No crystals, Oleoresin Nature |

### 3.3: Large Scale Production of High Purity Crystalline Lutein Ester from Marigold Shredded Flower

100 kg shredded marigold flower taken in an extractor and treated with ethanol in the weight ratio of flower to ethanol (1:5). The mixture was heated at 40 to 50° C. with stirring and extracted with specified volume of ethanol. The mixture was then filtered, and the residue re-extracted with ethanol until the ethanol solubles are completely extracted, 4 cycles of extraction were performed. The residual biomass was treated with acetone in the weight ratio of flower to acetone (1:4) at 40 to 45° C. Repeated extraction done with acetone to recover all the xanthophylls from the biomass. The acetone miscella concentrated to 2% concentration and kept for crystallization for 12 hrs at 10° C. The crystals of trans lutein esters settled were filtered out, dried under DHAD at 45 to 48° C. The total carotenoids estimated by UV-Vis and lutein esters measured by HPLC after saponification.

TABLE 3.2
Product profile of high purity lutein
ester from shredded marigold flower

| | % Yield from | | |
|---|---|---|---|
| Experiment | shredded flower | % Total Carotenoid | % Lutein ester |
| 1 | 0.64 | 87 | 81.8 |

Impurities (contaminants) generally seen in Marigold products are poly aromatic hydrocarbons, dioxins, nitrosamine, ethylene oxide etc.

TABLE 3.3
Impurity profiling of high purity lutein
ester from shredded marigold flower

| Parameters | Results |
|---|---|
| Protein % | 0.12 |
| Ash % | <0.1 |
| Poly cyclic aromatic hydrocarbons in ug/kg | Not detected |
| Benzopyrene in ug/kg | Not detected |
| Pesticides | Not detected |
| Dioxins, furans in pg/g | 0.488 |
| Melamine in mg/kg | <0.1 |
| Ethylene Oxide in ppb | <10 |
| Nitrosamine in mg/kg | <0.01 |

### Ethanol Extract (i.e. Polar Solvent Fraction)

Ethanol miscella from the experiment desolventised to an extract with polyphenols as the active component. The major polyphenol in the extract is quercetagetin.

TABLE 3.4
Polyphenols from the ethanol extract

| % Yield | % Polyphenols | % Quercetagetin |
|---|---|---|
| 1.8 | 34 | 24 |

The ethanol extract on purification using carbon treatment in ethanol medium yielded quercetagetin >90% purity.

TABLE 3.5
Isolation of quercetagetin from ethanol extract

| | % Yield | % Polyphenols | % Quercetagetin |
|---|---|---|---|
| Experiment 1 | 0.28 | 92.4 | 91.3 |

Oleoresin from the by-Product of Lutein Ester

The by-product obtained by de-solventising the mother liquor shows quality like conventional (hexane extracted) oleoresin. Yield of the by-product is 0.8 to 1.6% with Xanthophylls 12 to 16% having 68 to 74% trans lutein as shown in Table 3.6. This Oleoresin can be saponified to get free lutein >74% purity.

TABLE 3.6
Oleoresin from by-product of lutein ester

| | | | % |
|---|---|---|---|
| | | | Zeaxanthin |
| % Oleoresin Yield | % Xanthophyll | % Lutein by HPLC | by HPLC |
| 1.5 | 14 | 71 | 3.4 |

### Example 4: Extraction of Lutein Ester from Marigold Meal Using Isopropanol

Marigold meal (250 g) prepared using Example 1 was taken in a 5 L Erlenmeyer flask and was treated with isopropanol in the weight ratio of meal: isopropanol ranging from 1:2 to 1:8, as shown in Table 4. The mixture was heated at 55° C. with stirring and extracted with circulation of isopropanol for a period of 30 minutes. The mixture was then filtered, and the residue was re-extracted with isopropanol until the isopropanol soluble content was completely extracted, preferably 4 cycles of extraction. The isopropanol fraction was then concentrated until 5 to 10% of extractives to obtain an extract comprising lutein ester as shown in Table 4. The extract was then subjected to crystallization at 35° C. for 24 to 72 hours, preferably 48 hours, as shown in Table 4. The crystals of trans lutein esters that settled down were filtered out, dried under DHAD at 45 to 48° C.

### Observation and Results

The extraction method using isopropanol yielded lutein ester crystals in the range of 2 to 3% of the meal with a purity of trans lutein ester in the isolated crystals of 70 to 75% and 2 to 6% trans zeaxanthin ester (Table 4). Particularly, Experiment No. 2, which involved the use of 1:5 meal:isopropanol weight ratio and with crystallization carried out at 35° C. and 48 hours yielded 73.9% lutein ester crystals.

The mother liquor was concentrated to oleoresin with a yield of 20 to 22% in respect of the meal and xanthophyll content in the oleoresin was 5 to 8%. Trans lutein in this oleoresin was 65 to 70%. Particularly, 22.7% of oleoresin in respect of the meal, comprising 6.8% of xanthophylls. Table 4 illustrates working and non-working examples, wherein experiments 1 to 3 achieved satisfactory results. However, in experiments 4, 5 and 6 no crystals and oleoresin nature observed.

TABLE 4
Experimental conditions for high purity lutein ester extracted using isopropyl alcohol.

| | | | | | % | | | |
|---|---|---|---|---|---|---|---|---|
| | | | Crystallization | | Yield | | Lutein | Zeaxanthin |
| | Meal to | Total | condition | | Lutein | Total | ester | ester |
| Experiment | isopropanol | Extractives | Time | Temp | ester | Carotenoids | purity | purity |
| No. | Ratio | (%) | (hrs) | (° C.) | crystal | (%) | (%) | (%) |
| 1 | 1:2 | 5 | 24 | 35 | 2.5 | 76 | 71.1 | 3.2 |
| 2 | 1:5 | 10 | 48 | 35 | 2.7 | 79 | 73.9 | 3.4 |
| 3 | 1:8 | 8 | 72 | 35 | 2.4 | 77 | 72 | 3.1 |

| | | | | | |
|---|---|---|---|---|---|
| 4 | 1:2 | 10 | 10 | 35 | No crystals, Oleoresin nature |
| 5 | 1:12 | 10 | 48 | 35 | No crystals, Oleoresin Nature |
| 6 | 1:5 | 15 | 48 | 35 | No crystals, Oleoresin Nature |

### Advantages of the Present Disclosure

The present disclosure provides a highly efficient extraction method for obtaining extracts having and high purity crystalline lutein ester. Unlike conventional methods, the disclosed method does not involve hexane treatment of marigold meal/pellet and instead uses the marigold—flowers/meal/pellet directly in the extraction procedure using environment friendly solvents—ethanol and acetone; or isopropanol. It is an innovative process having lesser no. of unit operations and with higher xanthophyll recoveries in the crystalline lutein ester. The disclosed method is a simple solvent extraction process and does not involve multistage laborious solvent purification procedures. Therefore, the method can be scaled up for the use of high purity lutein ester crystals in pharmaceutical and dietary supplement compositions.

I/We claim:

1. A method for preparing lutein ester, comprising:
a) providing raw material selected from shredded marigold flowers, marigold meal, or combination thereof;
b) subjecting the raw material to extraction with at least one polar solvent selected from ethanol, methanol, or combination thereof, to obtain a polar solvent fraction and residual biomass;
c) subjecting the residual biomass to extraction with an extractant comprising acetone, to obtain an acetone fraction; and
d) concentrating the acetone fraction.

2. The method as claimed in claim 1, wherein said concentrating is performed to obtain an extract comprising 1 to 12 wt % extractives, preferably 1 to 5% extractives when the raw material is shredded marigold flowers, preferably 5 to 10% extractives when the raw material is marigold meal.

3. The method as claimed in claim 2, further comprising crystallizing the extract to obtain crystals comprising lutein ester, and mother liquor.

4. The method as claimed in claim 1, wherein the ratio of the raw material and polar solvent is in a range of 1:2 to 1:8.

5. The method as claimed in claim 1, wherein step (b) is performed at a temperature in the range of 30 to 60° C., with continuous circulation of the solvent for a period of 20 to 100 minutes.

6. The method as claimed in claim 1, wherein ratio of said residual biomass and extractant is in a range of 1:2 to 1:8.

7. The method as claimed in claim 1, wherein step (c) is performed at a temperature in a range of 25 to 52° C., with continuous circulation of the extractant for a period of 20 to 60 minutes.

8. The method as claimed in claim 3, wherein said crystallization is performed at a temperature in the range of 7 to 40° C., for a period of 8 to 60 hrs, preferably 10 to 30 hours.

9. The method as claimed in claim 3, wherein yield of said lutein ester is in a range of 0.25 to 2.5 wt % in respect of the raw material, preferably 1.1 to 2.5 wt % when the raw material is marigold meal, or preferably 0.25 to 1.0 wt % when the raw material is shredded marigold flowers.

10. The method as claimed in claim 3, wherein purity of lutein ester is at least 55 wt %, preferably at least 76 wt %, and wherein said crystals further comprises zeaxanthin ester in a range of 2 to 6 wt %.

11. The method as claimed in claim 3, further comprising concentrating said mother liquor to obtain an oleoresin, wherein the oleoresin comprises xanthophyll in a range of 12 to 16 wt %, of which 70 to 75 wt % is trans lutein and 3 to 6 wt % is zeaxanthin.

12. The method as claimed in claim 11, wherein the oleoresin is subjected to saponification to obtain free lutein of at least 74% purity.

13. The method as claimed in claim 1, wherein said polar solvent fraction comprises at least one polyphenol selected from quercetagetin, 6-hydroxy kaempferol, quercetin, patuletin, or mixtures thereof.

14. The method as claimed in claim 1, further comprising subjecting the polar solvent fraction to purification with carbon in ethanol to obtain quercetagetin.

15. A method for preparing lutein ester, comprising:
(a) providing raw material selected from shredded marigold flowers, marigold meal, or combination thereof;
(b) subjecting the raw material to extraction with an extractant comprising isopropanol, to obtain an isopropanol fraction; and
(c) concentrating the isopropanol fraction, and optionally followed by crystallization.

16. The method as claimed in claim 15, wherein the crystallization is performed to obtain crystals comprising lutein ester, and mother liquor.

17. The method as claimed in claim 15, wherein ratio of the raw material and extractant is in a range of 1:2 to 1:8, preferably 1:5.

18. The method as claimed in claim 15, wherein step (b) is performed at a temperature in the range of 50 to 60° C., with continuous circulation of the solvent for a period of 20 to 40 minutes, preferably 30 minutes.

19. The method as claimed in claim 15, wherein said crystallization is performed at a temperature in the range of 10 to 55° C., for a period of 5 to 90 hours.

20. The method as claimed in claim 15, wherein said concentrating is performed to obtain an extract comprising 1 to 12% extractives, preferably 5 to 12% extractives.

21. The method as claimed in claim 16, wherein yield of said lutein ester is in a range of 2 to 3 wt %, in respect of the marigold meal.

22. The method as claimed in claim 16, wherein purity of lutein ester is at least 55 wt %, and wherein said crystals further comprise zeaxanthin ester in the range of 2 to 6 wt %.

23. The method as claimed in claim 16, further comprising concentrating said mother liquor to obtain an oleoresin, wherein the oleoresin comprises xanthophyll in the weight range of 5 to 8 wt %, of which lutein is in the weight range of 70 to 75 wt % and zeaxanthin in the weight range of 3 to 6 wt %.

24. The method as claimed in claim 1, wherein said marigold meal is obtained by a process comprising silaging marigold flowers with at least one enzyme selected from cellulases, hemicellulases, cellobiohydrases, pectinases, galactomannanases, proteases, lipases, and combination thereof, preferably for a period of 5 to 10 days; and dehydrating to obtain marigold meal.

25. The method as claimed in claim 1, wherein step (b) and/or step (c) is repeated at least once, preferably 2 to 5 times.

26. The method as claimed in claim 3, wherein said crystals comprise protein at a weight range of 0 to 0.5%, ash at a weight range of 0 to 0.2%; poly aromatic hydrocarbons (PAH) in an amount of 0 to 2 ppb, benzopyrene in an amount of 0 to 0.5 ppb, pesticides in an amount of 0 to 0.01 ppm, dioxins in an amount of 0 to 0.5 pg/g, melamine in an amount of 0 to 0.1 mg/kg, ethylene oxide in an amount of 0 to 10 ppb, and nitrosamine in an amount of 0 to 0.01 mg/kg.

27. The method as claimed in claim 14, wherein said quercetagetin is of at least 90% purity.

28. The method as claimed in claim 1, wherein said concentrating is performed to obtain an extract comprising xanthophyll in the weight range of 15 to 30 wt %, lutein in the weight range of 70 to 80 wt %, and zeaxanthin in the weight range of 3 to 6 wt %.
